(19) United States
(12) Patent Application Publication
(10) Pub. No.: US 2022/0062042 A1
(43) Pub. Date: Mar. 3, 2022

(54) SYSTEM FOR THE APPLICATION OF EYEDROPS

(71) Applicants: Paul McGovern, JR., Henderson, NV (US); Marilyn McGovern, Henderson, NV (US)

(72) Inventors: Paul McGovern, JR., Henderson, NV (US); Marilyn McGovern, Henderson, NV (US)

(21) Appl. No.: 17/004,313

(22) Filed: Aug. 27, 2020

(51) Int. Cl. A61F 9/00; G02C 5/00

(52) U.S. Cl. CPC A61F 9/0026; G02C 5/001

(57) ABSTRACT

A system for introducing eye drops into the eyes of a user is disclosed. The system includes non-prescription sun glass frames. The glasses have a small circular opening the middle of each lens positioned above a user's open eye. In one embodiment, the opening an inner surface surrounding the opening includes inner threads. The liquid releasing end of a squeeze bottle of eyedrops may be placed securely in the application opening and drop applied with no spill or waste. An additional benefit of the sun glass design is to enable users who have recently had their eyes dilated to safely apply eye drops. The system is designed to reduce physiological issues associated with applying eyedrops.

### II. BACKGROUND OF THE INVENTION

### 1. Field of the Invention

The present invention relates to the application of eyedrops and, more particularly, to a system for the application of eyedrops that includes a bottle mounted to a pair of darkened lenses for the application of eyedrops on a user's eyes.

### 2. Description of the Related Art

Several designs for apparatuses for applying eyedrops have been designed in the past. None of them, however, include a system for introducing eye drops into the eyes of a user. The system includes non-prescription sun glass frames. The glasses have a small circular opening the middle of each lens positioned above a user's open eye. In one embodiment, the opening an inner surface surrounding the opening includes inner threads. The liquid releasing end of a squeeze bottle of eyedrops may be placed securely in the application opening and drop applied with no spill or waste. An additional benefit of the sun glass design is to enable users who have recently had their eyes dilated to safely apply eye drops. The system is designed to reduce physiological issues associated with applying eyedrops. It is known that the process of applying eye drops could often be and difficult process which induces a substantial amount of physiological stress on users. The view of the bottle from a user's eye may induce the stress and makes it difficult for applying eye drops. Therefore, these is a need for a system for applying eye drops which includes a dark sunglass to omit the view of the bottle from the user. The lenses further include an opening which receives the tip of an eye drop bottle to aid a user in applying eye drops while simultaneously reducing stress.

Applicant believes that a related reference corresponds to U.S. Pat. No. 3,446,209 issued for a device for facilitating self-introduction of medicinal drops in eyes. The device includes a shield with positioning means thereon which engage the head of a user. Applicant believes that another related reference corresponds to U.S. Pat. No. 4,183,355 issued for a frame for therapeutic self-administration of medication to the eyes and comprised of transparent right angularly related slides with normal slots therethrough for the reception of guide-adjustment-lock means. However, the cited references differ from the present invention because the cited references fail to disclose the dark lenses which are mounted to the frame. The dark lenses obscure the application bottle from a user's eyes in order to reduce the physiological stress associated with the application of eye drops. The application bottle is then mounted to an opening of each of the lenses which will position the bottle above a user's eyes for easy application.

Other documents describing the closest subject matter provide for a number of more or less complicated features that fail to solve the problem in an efficient and economical way. None of these patents suggest the novel features of the present invention.

### III. SUMMARY OF THE INVENTION

It is one of the objects of the present invention to provide a system for the application of eye drops which reduces the physiological stress associated with applying eye drops.

It is another object of this invention to provide a system for the application of eye drops which includes darkened lenses to obscure vision of an application bottle to reduce stress for a user.

It is still another object of the present invention to provide a system for the application of eye drops which includes a mounting means for an application bottle to place the bottle directly over a user's eye for easy application.

It is yet another object of this invention to provide such a device that is inexpensive to implement and maintain while retaining its effectiveness.

Further objects of the invention will be brought out in the following part of the specification, wherein detailed description is for the purpose of fully disclosing the invention without placing limitations thereon.

### IV. BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 represents an operational isometric view of application system 10 depicting a user applying eye drops in accordance to an embodiment of the present invention.

FIG. 2 shows an isometric view of frame assembly 20 in accordance to an embodiment of the present invention.

FIG. 3 illustrates an isometric view of bottle assembly 40 in accordance to an embodiment of the present invention.

FIG. 4 is a representation of an isometric view of frame assembly 20 depicting inner threads in accordance to another embodiment of the present invention.

FIG. 5 shows an isometric view of bottle assembly 40 depicting outer threads in accordance to an embodiment of the present invention.

FIG. 6 shows an isometric view of bottle assembly 40 mounted to frame assembly 20 in accordance to an embodiment of the present invention.

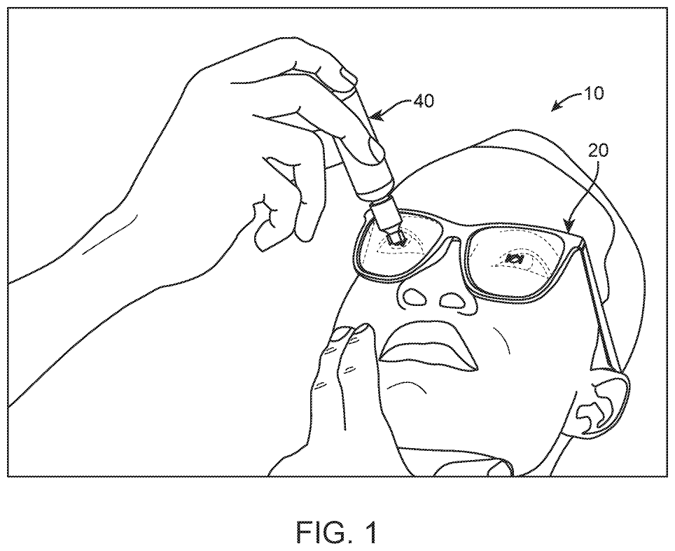

### V. DETAILED DESCRIPTION OF THE EMBODIMENTS OF THE INVENTION

Referring now to the drawings, where the present invention is generally referred to with numeral 10, it can be observed application system 10 for applying eye drops which basically includes a frame assembly 20 and a bottle assembly 40.

Frame assembly 20 includes a frame 22 configured to resemble the frame of traditional sunglasses. In one embodiment, frame 22 is provided as a sunglasses frame having two extending hinged connecting rods that are mounted onto a user's ears to securely mount the frame 22 to a user's head. Furthermore, frame 22 may include two pairs of enclosed perimeter areas. In traditional embodiments, the enclosed perimeter areas would be configured to receive traditional sunglass lens which provide protection to a user's eyes from sun rays that may cause harmful damage to the user's eyes. In other embodiments, the enclosed perimeter areas may be provided as partially enclosed perimeter areas which receive lenses therein to be supported. Other embodiments may also feature a frame 22 which is configured to only hold one lens, also known as a monocle frame.

In the present embodiment, frame 22 include lenses 24 mounted within the enclosed perimeter areas. The lenses 24 for the purposes of the provided specification are defined as a lens item which completely obscures the vision of a user wearing frame 22. Traditional lenses are defined as having a particular set of lenses to provide benefit to a user which may include prescription lenses to aid a user's vision or dark lenses to aid a user in blocking out sun rays to protect vision. However, in the present invention, lenses 24 are dark lenses which include a suitable tint which completely blocks the vision of a user. The purposes of lenses 24 being dark is such to reduce the physiological stress induced when observing bottle assembly 40 when applying eye drops. Lenses will prevent a user from seeing bottle assembly 40 thereby reducing physiological stress. Lenses 24 further includes an opening 25 located on a center portion on each of said lenses 24. In one embodiment, opening 25 is provided as a suitable diameter which receives bottle assembly 40 further, opening 25 may be provided such that the opening is located directly over a user's cornea to then receive an eye drop relief solution. In one embodiment, opening 25 is provided as a circular shape, however, any suitable shape to receive bottle assembly 40 may be used.

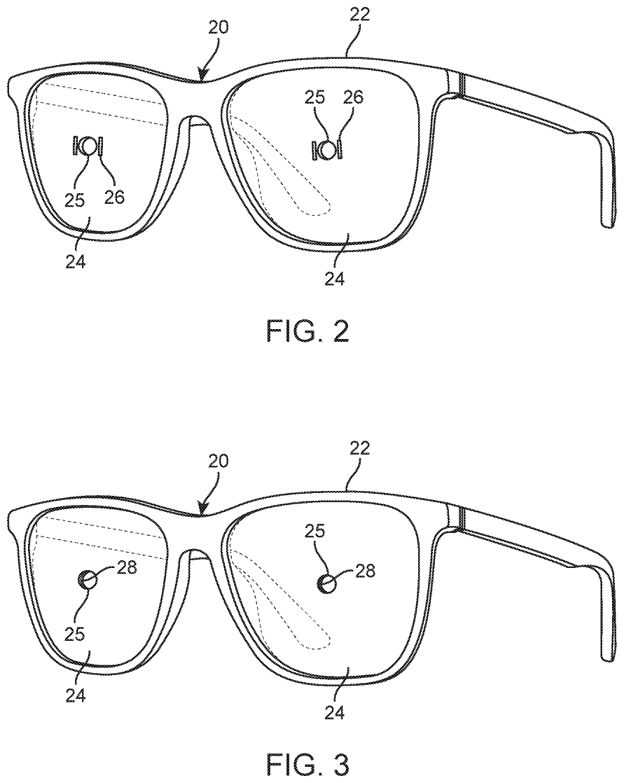

In one embodiment, as observed in FIG. 2, frame assembly 20 includes attachment members 26 mounted along a perimeter of opening 25. In one implementation, attachment members 26 are provided as a snap lock member which is configured to lock onto bottle assembly 40 when mounted therein. In another implementation, attachment members 26 may be provided as hook fasteners configured to lock onto bottle assembly 40. In yet another implementation, attachment members 26 may be provided in the form of an adhesive material to attach onto bottle assembly 40.

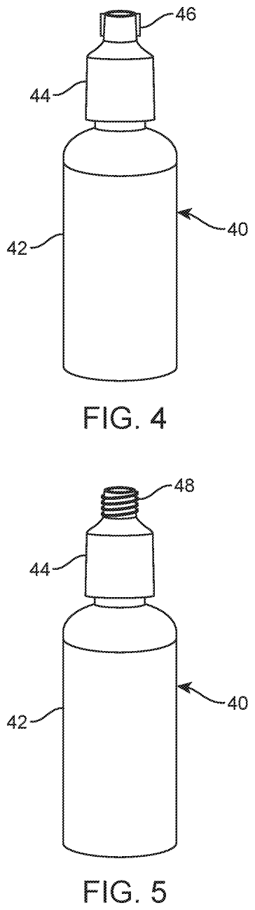

In another embodiment, as observed in FIG. 4, frame assembly 20 includes inner threads 28. Inner threads 28 may be located along an inner surface which surrounds a perimeter of opening 25. Inner threads are provided as a threaded structure which may be suitably received by outer threads to form a secure attachment means. Furthermore, as observed in FIG. 4, inner threads 28 are provided for each of said opening 25 of lenses 24. Embodiments of the present invention may also feature a combination of attachment members 26 and inner threads 28.

Bottle assembly 40 includes a container 42 made of a malleable plastic material that is configured to be squeezed by a user to dispense a product. In one embodiment container 42 is provided has having a cylindrical shape with an interior portion. In the present embodiment, container 42 holds an eye drop relief product therein to provide relief to a user's eye in need. The eye drop relief product or solution may provide relief to a user suffering from allergies or a user in need of relief from dry eyes which is usually a common symptom of contact lenses users. Furthermore, the eye drop solution may be a composition made of polyethylene glycol, polyvinyl alcohol, propylene glycol, carboxymethylcellulose, povidone, glycerin, and mineral oil. Bottle assembly 40 further includes a tip 44 mounted to a top end of container 42. In one embodiment, tip 44 is provided as a conical tapering upwardly from said container to a top end. Further, the top of tip 44 may be provided as having a circular opening from which the eye drop relief solution is dispensed therefrom. The top end of tip 44 must have a suitable diameter which cooperates with a diameter provided for opening 25.

In one embodiment, as observed in FIG. 3, tip 44 of bottle assembly 40 is provided with receiving members 46. In the present embodiment, receiving members 46 are structural members that receive attachment members 26 to form a secure attachment. In one embodiment, receiving members 46 are provided as aperture members which receive the snap lock member of attachment members 26. In another embodiment, receiving members 46 are provided as loop members which receive the hook members of attachment members 26 forming a hook and loop attachment system. In yet another embodiment, receiving members 46 are surface members which receive an adhesive material of attachment members 26.

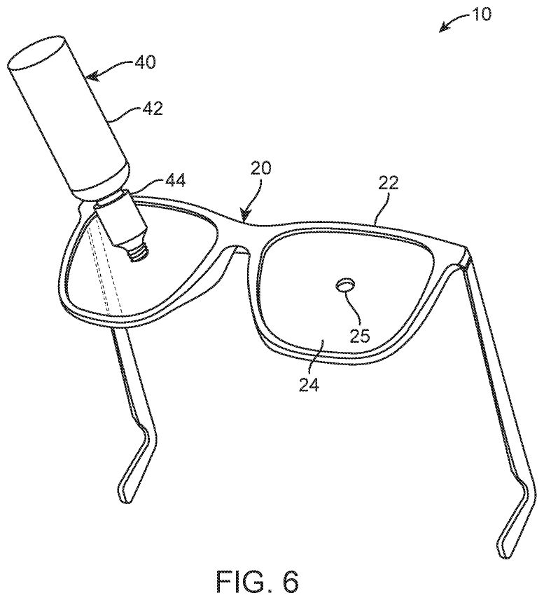

In another embodiment, as observed in FIG. 5, tip 44 may be provided with outer threads 48 mounted along the outer surface area of tip 44. Outer threads 48 is a threaded structure which is cooperates with inner threads 28 to be securely mounted thereon. In one embodiment, outer threads 48 entirely wrap around tip 44. Embodiments of the present invention may also include a combination of outer threads 48 and receiving members 46.

To utilize application system 10, a user will mount frame 22 to their head placing the lenses 24 right in front of their eyes. While having the frame 22 mounted, the user may then attach bottle assembly 40 to frame assembly 20. This may be accomplished by securing tip 44 to attachment members 26 by receiving members 46. The attachment could also be achieved by coupling outer threads 48 of tip 44 into inner threads 28 of lenses 24. Once both the frame assembly 20 and the bottle assembly 40 are suitably attached, the user may then tilt their head rearwards so as to look upwardly. The opening 25 of the frame 22 and there by the tip 44 is positioned directly above a user's cornea. The dark nature of lenses 24 will obscure the vision of bottle assembly 40 by a user. As a result, the physiological stress induced from placing eye drops is reduced as the bottle will not be in view by the user. The user may then simply squeeze container 42 to dispense the eye drop solution directly onto the cornea of a user.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

What is claimed is:

1. A system for the application of eye drops, comprising:
a) a frame assembly including a frame having at least one lens mounted thereon, wherein aid frame is adapted to be mounted to the head of a user, wherein said at least one lens is a dark lens which completely obscures a user's vision, said at least one lens having an opening and an inner surface surrounding said opening, inner threads located on said inner surface; and
b) a bottle assembly including a container and a tip, wherein said container includes an eye drop solution therein, said tip including an outer surface including outer threads to be threadably received by said inner threads of said inner surface.

2. The system for the application of eye drops of claim 1 wherein said frame includes two hinged rods.

3. The system for the application of eye drops of claim 1 wherein said opening is located on a center portion of said at least one lens.

4. The system for the application of eye drops of claim 1 wherein said at least one lens includes attachment members mounted to a perimeter of said opening.

5. The system for the application of eye drops of claim 4 wherein said attachment members are adhesive members.

6. The system for the application of eye drops of claim 4 wherein said outer surface of said tip includes receiving members to receive said attachment members.

7. The system for the application of eye drops of claim 6 wherein said receiving members are surface adhesive receiving members.

8. The system for the application of eye drops of claim 1 wherein said tip has a conical shape.

9. The system for the application of eye drops of claim 1 wherein said tip includes an open top end.

10. A system for the application of eye drops, comprising:
a) a frame assembly including a frame having two extending hinged rods adapted to be mounted to a user's ears, said frame including a first fully enclosed perimeter area and a second fully enclosed perimeter area, wherein a first lens is mounted within said first fully enclosed perimeter area, wherein a second lens is mounted within said second fully enclosed perimeter area, wherein said first lens and said second lens each include an opening located at a center portion, wherein said first lens and said second lens includes an inner surface surrounding said opening, wherein said inner surface includes inner threads extending thereon, said first lens and said second lens further including attachment members surrounding a perimeter of said opening, wherein said attachment members are hook attachment members, wherein said first lens and said second lens are dark lenses which completely obscure said user's vision; and
b) a bottle assembly including a container housing an eye drop solution therein, wherein said container is made of a malleable plastic material, a tip with an open top end mounted to a top of said container being in fluid communication with said container, wherein said tip is a conical member with an outer surface, wherein said outer surface of said tip includes outer threads extending thereon, wherein said outer threads are mounted to said inner threads of said inner surface to form a secure connection, said tip further including receiving members to receive said attachment members, wherein said receiving members are loop receiving members.
